(19) United States
(12) Patent Application Publication
(10) Pub. No.: US 2012/0109202 A1
(43) Pub. Date: May 3, 2012

(54) INTERSEGMENTAL MOTION PRESERVATION SYSTEM FOR USE IN THE SPINE AND METHODS FOR USE THEREOF

(75) Inventors: Ryan M. Kretzer, Baltimore, MD (US); Bryan W. Cunningham, Baltimore, MD (US); Jeffrey Gordon, Saratoga Springs, NY (US)

(73) Assignees: NEURAXIS LLC, Baltimore, MD (US); The Johns Hopkins University, Baltimore, MD (US)

(21) Appl. No.: 13/284,595

(22) Filed: Oct. 28, 2011

Related U.S. Application Data
(63) Continuation-in-part of application No. 13/098,365, filed on Apr. 29, 2011.
(60) Provisional application No. 61/329,865, filed on Apr. 30, 2010.
(60) Provisional application No. 61/386,229, filed on Sep. 24, 2010.
(60) Provisional application No. 61/430,140, filed on Jan. 5, 2011.

(51) Int. Cl. A61B 17/70

(52) U.S. Cl. 606/248

(57) ABSTRACT

Medical devices and kits for dynamically stabilizing or preserving motion in a spine and limiting adjacent, non-adjacent, or isolated segment degeneration of the spine while providing a controlled, determinable range of motion at the treated site, as well as methods for surgical use of the system.

### CROSS REFERENCE TO RELATED APPLICATIONS

This application is a continuation-in-part application of U.S. application Ser. No. 13/098,365, filed Apr. 29, 2011, which claims the priority benefit under 35 U.S.C. §119(e) of U.S. Provisional Application No. 61/329,865, filed Apr. 30, 2010, U.S. Provisional Application No. 61/386,229, filed Sep. 24, 2010, and U.S. Provisional Application No. 61/430,140, filed Jan. 5, 2011. Each of the foregoing applications is hereby incorporated by reference in its entirety.

### FIELD OF INVENTION

The invention relates to devices, systems and methods for use in treating spinal degeneration. More particularly, the invention relates to devices for use in stabilization and preservation of motion within a degenerated spinal column, including systems and methods for posterior ligamentous stabilization and reconstruction in the spine.

### BACKGROUND

Pedicle screw instrumentation in the spine has gained prominence in recent years due to the superior biomechanical properties provided by three column fixation. While these biomechanical advantages have improved construct stability over the operative spinal segment, the same factors that contribute to motion reduction also have been implicated in the progression of adjacent segment degeneration. Such adjacent segment degeneration is especially true with increasing construct length and when mild degenerative changes already exist at the supra- and/or infra-adjacent segments.

Efforts to address adjacent segment degeneration have included use of devices employing a pedicle screw-based design, with a rod or cord disposed in between the pedicle screw fixation points. One problem with this design has been that motion is decreased at the desired segment in a non-physiologic manner, which makes these devices prone to failure.

### SUMMARY

The presently disclosed subject matter provides a posterior-based intersegmental motion preservation system for use in the spine. The system includes, in all embodiments, elastic elements which stabilize the area of the spine treated while preserving a desirable range of motion, preferably a substantially physiologic range of motion.

According to one aspect, the system provides a device comprising more than one elastic tensile member disposed opposite one another in the spinal region to be treated via attachment to either adjacent spinous processes or directly from a spinal instrumentation construct (e.g., crosslink, rod, pedicle screw, laminar screw, lateral mass screw, etc.) to an adjacent spinous process(es).

In a further aspect, the intersegmental motion preservation system provides a device which comprises a single elastic tensile member in the spinal region to be treated via attachment to either an adjacent spinous process or directly from a spinal instrumentation construct (e.g., crosslink, rod, pedicle screw, laminar screw, lateral mass screw, etc.) to an adjacent spinous process.

In particularly preferred embodiments, the elastic tensile members are elastic and provide a graded resistance to spinal motion by physiologically stiffening the posterior ligamentous complex. Advantageously, the range of motion provided by use of the invention is greater than achievable by spinal fusion up to the clinically indicated limit for the patient treated (e.g., a patient whose spinal column has been treated or damaged at a different site may need a more limited range of motion to prevent further injury than one whose only impairment is treated by use of the invention). Most advantageously, the range of motion provided by use of the invention is substantially physiologic compared to spinal fusion.

The device of the invention also can be implanted in a patient in a quick and efficient manner through a minimally invasive approach, thereby limiting further destabilization of the adjacent segment. In these ways, the invention provides a crucial tool for a spine surgeon to limit adjacent segment or non-adjacent segment range of motion and potential degeneration following operative fixation at all levels of the spine while providing for a substantially physiologic range of motion around the treated area.

In those respects, the invention also provides methods for (i) stabilizing adjacent bones; (ii) connecting adjacent vertebral levels; and/or (iii) treating kyphosis, e.g., proximal or distal junction kyphosis, or adjacent segment/non-adjacent segment degeneration (disc/facet degeneration or listhesis) in a subject in need of treatment thereof, through delivery of the system of the invention to the subject's spine.

To those ends, the one or more elastic tensile members of the devices of the inventive system stretch on application of tensile force generated by flexion, axial rotation, or lateral bending of a subject's spine around the treated region then return to their original configuration on release of the applied force. The tension on the connecting members and their stiffness can be varied as necessary to stabilize the spine without allowing it more than a range of motion advisable for the condition of the treated region.

To enable ready use of the invention in treating spinal degeneration, the system of the invention is preferably provided as a surgical kit including a device as disclosed herein, optionally including a selection of elastic members for use in patients of differing sizes and in a variety of conditions, tools for use in implantation of the device, and user instructions for reference by the surgeon.

Certain aspects of the presently disclosed subject matter having been stated herein above, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1 is a schematic showing a posterolateral view of one embodiment of the presently disclosed device comprising a pair of opposing elastic members (bands) 100 and 100′ each having a first and second end disposed in the same plane for attachment to a spinous process 10;

FIG. 2 is the device shown in FIG. 1 where the elastic members 100 and 100′ have been stretched by separation of adjacent spinous processes 10;

FIG. 3 is a schematic showing an exploded view of the device of FIG. 1 showing a pair of articulating attachment elements comprised of male connector 152 for interlocking joiner to female connector 160 through bores 12 in spinous processes 10;

FIG. 4 is a schematic showing a posterolateral view of one embodiment of the presently disclosed device comprising a pair of opposing connecting elastic members 114 and 114′ (bands), each having a first and second end disposed in the same plane for attachment to a spinous process 10 between which is an undulating, deformable surface;

FIG. 5 shows the device of FIG. 4 where the elastic members 114 and 114′ have been stretched by separation of adjacent spinous processes 10;

FIG. 6 is a schematic showing an exploded view of the device of FIG. 4;

FIG. 7a is a schematic showing a posterolateral view of one embodiment of the presently disclosed device comprising an elastic member 200 which contains within it a cord which acts as a stiffener and/or displacement limiter, and which has a first and second end disposed in the same plane for attachment to spinous processes 10;

FIG. 7b is a schematic of the elastic member 200 shown in FIG. 7a shown in a stretched configuration, thereby showing the action of cord 204;

FIG. 8a is a schematic showing a posterolateral view of one embodiment of the presently disclosed device comprising an elastic member 300 which is in the form of a spring having a first and second end disposed in the same plane for attachment to spinous processes 10;

FIG. 8b is a schematic of the elastic member 300 shown in FIG. 8a shown in a stretched configuration;

FIG. 9 is a schematic showing a posterolateral view of one embodiment of the presently disclosed device comprising a pair of opposing elastic members 400 and 400′ which comprise a pair of flattened, elastic tubes, each with a first and second end disposed in the same plane for attachment to spinous processes 10;

FIG. 10 is a schematic showing a posterolateral view of one embodiment of the presently disclosed device comprising a pair of opposing elastic members 500 and 500′ in the form of a pair of elastic cords, each having a first and second end disposed in the same plane for attachment to spinous processes 10;

FIG. 11 is a schematic showing a posterolateral view of one embodiment of the presently disclosed device comprising a pair of opposing elastic members 600 and 600′ in the form of a pair of tethers, each having a first and second end disposed in the same plane for attachment to spinous processes 10;

FIG. 12 is a schematic showing a posterolateral view of one embodiment of the presently disclosed device comprising a pair of opposing elastic members 110 and 110′ each having means for attachment to more than two spinous processes 10;

FIG. 13 is a schematic showing a posterolateral view of one embodiment of a male/female connector attachment mechanism of the disclosed device used to attach the elastic members to one or more spinous processes 10;

FIG. 14 is a schematic showing one embodiment of the presently disclosed device comprising an elastic member 603 attached to a pair of hooks 656 for attachment to a patient's spine.

FIG. 15 is a schematic showing a posterior view of three vertebrae and showing one embodiment of the presently disclosed device in which a pair of elastic members 702 and 702′ are attached to rods 700 and 700′ and to a spinous process 10;

FIG. 16 is a schematic showing a posterior view of three vertebrae and showing one embodiment of the presently disclosed device in which an elastic member 703 is attached to a crosslink 786 and to a spinous process 10;

FIG. 17 is a schematic showing a posterior view of one embodiment of the presently disclosed device in which a pair of elastic members 800 and 800′ are attached to a pair of spinous processes 10 with a pair of clamps 820;

FIG. 18 is an exploded view of the alternative embodiment of the device shown in FIG. 17;

FIG. 19 is a schematic showing a posterolateral view of an alternative embodiment of the device wherein a single elastic member 100 is attached to the spinous processes 10;

FIG. 20 is an exploded view of the alternative embodiment of the device shown in FIG. 19;

FIG. 21 depicts a posterior view of a posterior ligamentous stabilization and reconstruction device 1000 according to another embodiment of the invention, the device positioned in which an elastic member 1002 is arranged to be attached to rods 1200 and 1200′ via a crosslink (not shown) and to a spinous process 10;

FIG. 22 illustrates the device 1000 shown in FIG. 21 when attached to a portion of a subject's spinal column;

FIG. 23 depicts an exploded perspective view of the device 1000 shown in FIGS. 21-22 including crosslink 1100 and multiaxis connectors 1120;

FIG. 24a depicts an assembled perspective view of the device 1000 shown in FIG. 23;

FIG. 24b depicts a side cross-sectional view of the spinous process attachment portion of the device 1000 of FIGS. 21-24a according to an example embodiment;

FIG. 25 depicts an exploded perspective view of the device 1000 of FIGS. 21-24b;

FIG. 26 depicts an assembled perspective view of the device 1000 of FIG. 25;

FIG. 27 depicts a perspective view of a posterior ligamentous stabilization and reconstruction device according to another embodiment of the invention, the device including two separate elements 2000, 2001;

FIG. 28 depicts a perspective view of the crosslink 1100 and multiaxis connector 1120 of FIGS. 22, 23, and 24a and as coupled to rod 1200;

FIG. 29 depicts a cross-sectional view of the multiaxis connector 1120 secured to the crosslink 1100 and rod 1200, the rod 1200 and crosslink 1100 extending substantially normal to one another; and

FIGS. 30 and 31 depicts several possible embodiments of a tool constructed and configured to form a bore through a spinous process(es) of a subject.

### DETAILED DESCRIPTION

The presently disclosed subject matter now will be described more fully hereinafter with reference to the accompanying Figures, in which some, but not all embodiments of the presently disclosed subject matters are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

All publications and other printed materials referenced herein are incorporated herein by this reference.

According to embodiments of the invention, the device may provide a graded resistance to spinal motion by physiologically stiffening or replacing the posterior ligamentous complex with an intersegmental motion preservation system including one or more elastic tensile members. More particularly, the presently disclosed device and methods may be used to stiffen the spinal segment in a physiologic manner by recapitulating the supraspinous/interspinous ligament and ligamentum flavum complex that exists in vivo, or to replace part or all of the ligamentous complex. To this end, the device provides a determinable graded tensile resistance that responds to the force applied to a connection element of the device, which resumes its baseline shape once the applied load is removed. Advantageously, the range of motion provided by use of the invention is greater than achievable by spinal fusion up to the clinically indicated limit for the patient treated (e.g., a patient whose spinal column has been treated or damaged at a different site may need a more limited range of motion to prevent further injury than one whose only impairment is treated by use of the invention). Most advantageously, the range of motion provided by use of the invention is substantially physiologic compared to spinal fusion.

Another feature of the presently disclosed device is that it can be implanted through either a minimally invasive or open approach and does not require operative dissection over the facet joints or disruption of the supraspinous/interspinous ligament complex. The one or more bands of the device also provide a physiologic stiffening of the posterior tension band of the spine in flexion, axial rotation, and lateral bending loading that directly counteracts the most common failure modes of spinal segment degeneration (kyphosis or listhesis due to hypermobility).

In those embodiments of the inventive device which employ one or more elastic tensile members, as described in more detail herein below, the tensile member may each be a band, spring, tube, rod or similar structure, provided in a variety of lengths depending on patient size and region of the spine. Once implanted between the spinous processes or between a spinal instrumentation construct (e.g. crosslink, rod, pedicle screw, laminar screw, lateral mass screw, etc.) and an adjacent spinous process, the one or more bands elongate under application of physiological loads and then resume their baseline shape once the load is removed.

The elongation capacity of each elastic tensile member can be optimized by varying its material, geometry and/or stiffness; i.e., from a low stiffness providing significant flexibility allowing for spinal motion to substantially physiologic degrees, to a high stiffness allowing for little or no motion around the treatment site.

In one embodiment, each elastic tensile member is secured to adjacent spinous processes at the site of treatment by a pair of connection elements provided in, through, over, or around the spinous processes at opposing ends of each elastic tensile member. Alternatively, one end of the elastic tensile member can be attached directly to spinal instrumentation (e.g. crosslink, rod, pedicle screw, laminar screw, lateral mass screw, etc.) while the other end of the elastic tensile member attaches to an adjacent spinous process. The connection elements can be, for example, articulating male and female pairs formed of a biocompatible metal or rigid polymer (such as polyether ether ketone [PEEK] to avoid MRI artifact at both ends). The connection elements may also have a coating, such as hydroxyapatite or other material or surface modification, to improve osteointegration or adherence to the spinous process. The male connector preferably has a head or base to retain the connection element, to provide a bone in growth surface, and to provide a surface for interaction with a surgical instrument, such as a wrench or inserter. Each connection element pair is provided in a variety of lengths to fit the dimensions of the treatment site (e.g., individual spinous process width).

In one aspect, a retaining ring non-rotatably secures the elastic tensile member(s) to the spinous process. Sliding connections such as cotter pins, lynch pins, or clips could also be used. In a further aspect, however, the male connector and female connector can be made rotatably connected, such as a threaded connection or a spiral locking ring. In yet a further embodiment, a single connecting member may be employed for attachment of the elastic tensile member, such as a cotter pin, suture, cable, wire, or an open ring that would then be crimped closed.

For implantation of the device at a spinous process, a hole may be made through a spinous process using a drill, awl, or other mechanism. The width of the spinous process is measured to choose the appropriate length connector that will lock securely without extending excessively beyond the spinous process. The distance between adjacent spinous processes (if more than one is used to anchor the device) or, for anchoring to implanted spinal instrumentation (e.g. a crosslink, rod, pedicle screw, laminar screw, lateral mass screw, etc.) and an adjacent spinous process, is measured at neutral position. The measurement is used to select the appropriately sized elastic tensile members, which members are chosen to provide a suitable stiffness as herein described.

The elastic tensile members are then attached to one or more spinous processes and subsequently locked in place. Once locked, the elastic tensile member(s) and connecting member(s) combine to form the final device, supporting and stiffening the supraspinous/interspinous ligament complex without disrupting the posterior ligamentous complex. In cases of laminectomy and/or resection of the posterior ligamentous structures (supraspinous ligament and/or interspinous ligament and/or ligamentum flavum and/or facet capsular ligaments), the device will serve to reconstruct the posterior ligamentous complex by attachment to the adjacent level spinous process(es) and spinal instrumentation (e.g. crosslink, rod, pedicle screw, laminar screw, lateral mass screw, etc.). Finally, the incision is closed in standard fashion.

Depending on the impairment, the stiffness of the device can be determined by choice of various lengths, widths, thicknesses or diameters as well as materials of the elastic members in order to allow anywhere from a low to high degree of tensile stiffness: ranging from 0.1 N/mm to 1,000 N/mm. For example, the tensile stiffness may be less than 1,000, 900, 800, 700, 600, 500, 400, 300, 200 or 100 N/mm. Generally, suitable materials for use in the device will be biocompatible ones including: biocompatible polymers such as nylon, PEEK, silicone, urethanes, aramids, polyethylenes and polypropylenes, as well as metals such as titanium and its alloys, stainless steel and cobalt chrome alloys, composites like carbon fiber, combinations of the previously mentioned materials, such as carbon fiber reinforced PEEK and other materials with properties meeting the foregoing criteria will be known to or readily ascertainable by those of ordinary skill in the art. The materials selection is most preferably made to provide a range of motion that is substantially equivalent to physiologic (or slightly less, as clinical requirements demand). In general, materials at the lower range of tensile stiffness will provide a greater range of motion, while stiffer ones will provide for a more controlled and delimited range of motion.

The device can be placed either above or below an existing spinal construct, or can be placed at a non-adjacent level that shows degenerative changes and is worrisome for further progression of degeneration in the future. One of ordinary skill in the art would appreciate that although the presently disclosed device is suited for the supra- or infra-adjacent segment above or below a spinal construct to reduce the incidence of proximal/distal junction kyphosis (PJK/DJK) or adjacent segment degeneration, the device can be placed at any level in the spine, including above or below a spinal construct or in the absence of prior spinal instrumentation. In other embodiments, the presently disclosed device also could be placed at a non-adjacent segment that has early signs of degeneration to delay further progression of spinal disease.

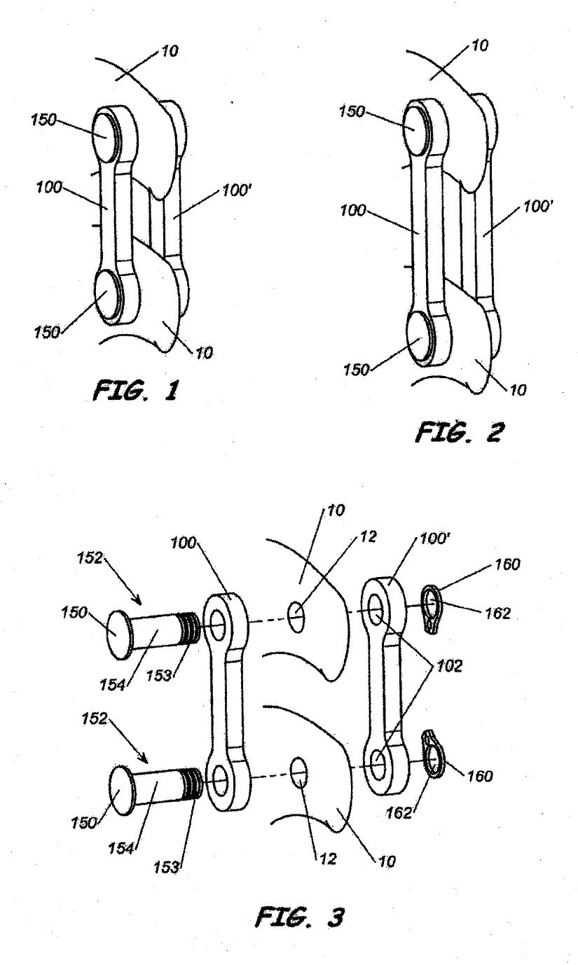

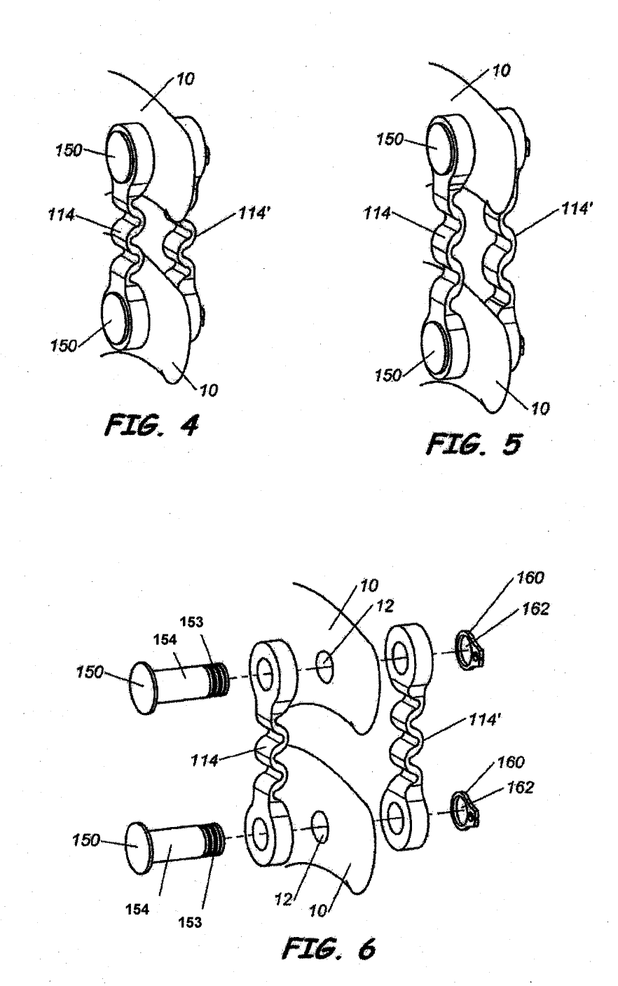

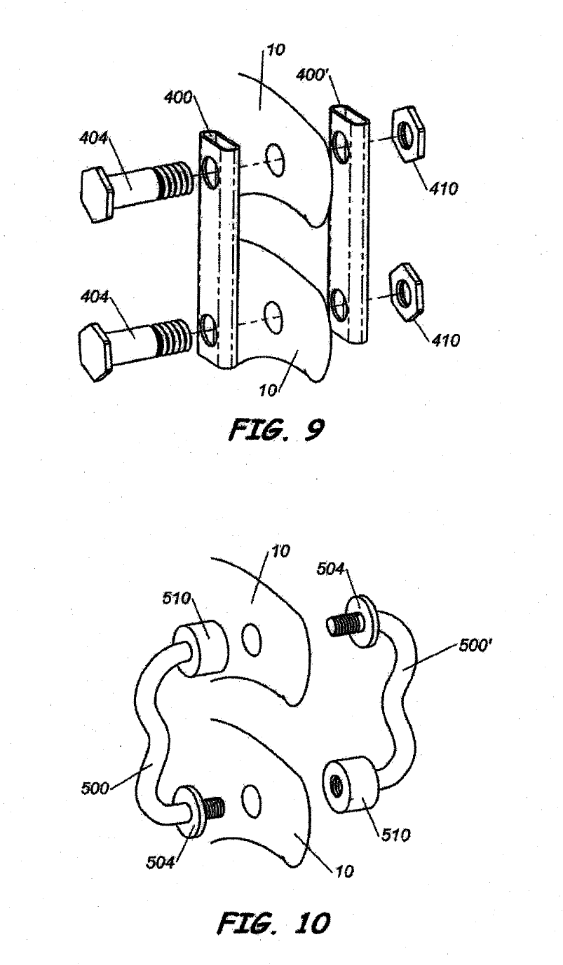

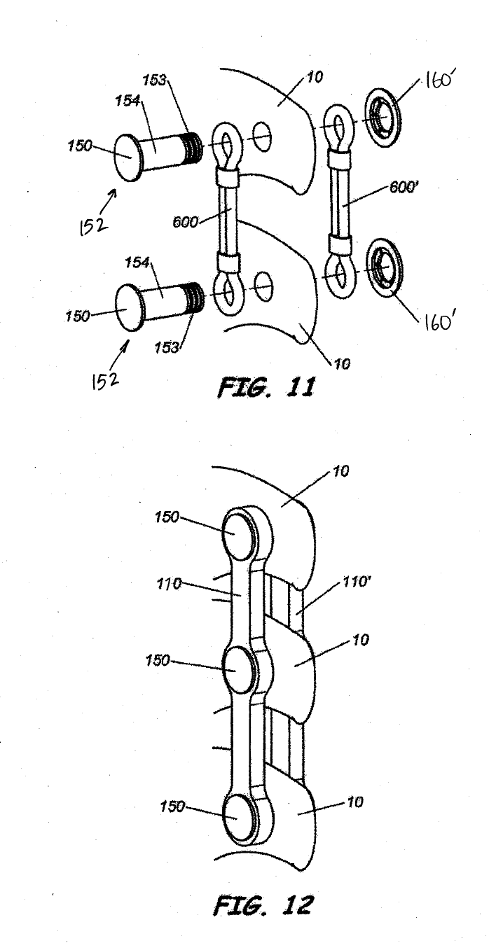

Referring now to FIGS. 1-3, one embodiment of the presently disclosed subject matter comprises two elastic members 100 and 100′, each having a first end and a second end for attachment to two spinous processes 10 by means of a male connector 152 and a corresponding female connector 160. As shown, elastic members 100 and 100′ are elastic bands, however the elastic members may be one or more other structures, such as a cord (FIGS. 10 & 11), a spring (FIGS. 8a & 8b), a band (FIGS. 4-6), a tether, a strap, a belt, a tube (FIG. 9), a wire, a tape, a cable, a suture, and the like. Referring to FIG. 3, male connector 152 and female connector 160 are complementary in that they can be articulately connected or united to form an interlocking joint. Male connector 152 comprises a head 150, a shaft 154, and one or more grooves 153. Shaft 154 is sized for insertion through a bore 162 of female connector 160, which is in turn sized to correspond in diameter to the bore 12 to be drilled or otherwise formed into spinous process 10.

Female connector 160 comprises bore 162, which is adapted to articulately connect, e.g., interlock, with one or more grooves 153 provided on shaft 154 of male connector 152. When interlocking elastic members 100 and 100′ are attached, joining of the male and female connectors by disposing bore 162 over a corresponding groove 153 in male connector (adapter) 152 creates a continuous connecting member on each side of the spinous process pair without disrupting the supraspinous/interspinous ligament complex. Connectors 152 and 160 are shown in the drawings as being generally cylindrical or round in shape. It will be understood, however, that other shapes may be utilized so long as the adapters can be connected through the bone (e.g., through the spinous process).

An alternative configuration of an elastic connecting member is shown in FIGS. 4-6. As shown, elastic members 114 and 114′ are undulating bands. The undulations may provide the device additional capacity for motion in response to application of physiologic force, and allow elastic members 114 and 114′ to be manufactured from a relatively stiff biocompatible material such as, for example, titanium alloy or stainless steel. Elastic members 114 and 114′ would therefore elongate by flexion of the undulations much like leaf springs.

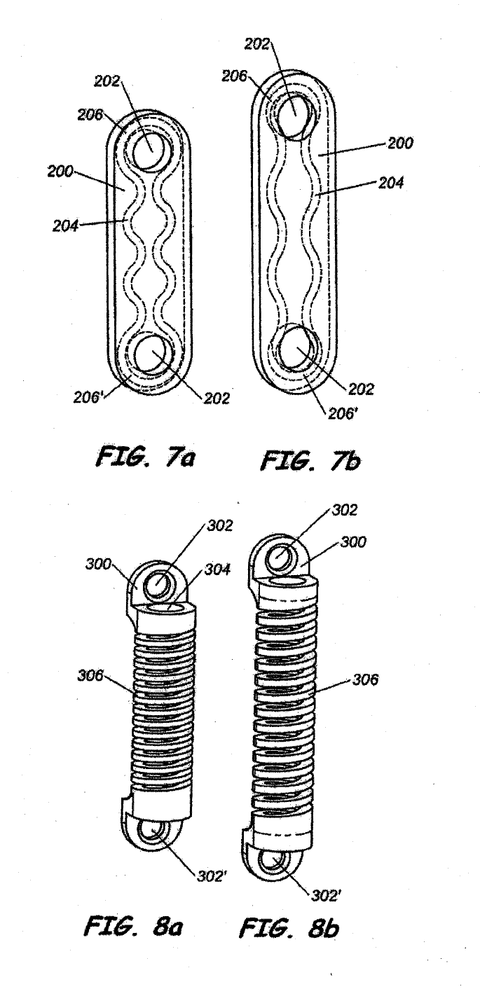

An additional alternative for use as connecting members in the device of the invention is shown in FIGS. 7a & 7b. As shown, a band 204 may be embedded within an elastic member 200. The function of elastic member 200 is much the same as described for elastic members 100 and 100′, with the addition of band 204 to stiffen the device or to act as a displacement limit for the device. Opposing open ends 202 of the elastic member 200 facilitate connection to spinous processes 10 by, for example, male/female connectors 152, 160. FIG. 7b shows the same device as FIG. 7a, where the device has been stretched.

Another alternative for use as an elastic member in the device of the invention is depicted in FIGS. 8a & 8b. As shown, the elastic member can be in the form of an expandable helical spring 300 with a bore 304 to form a helical flexure 306. Such a spring may be produced, for example, either by helically bending a wire (not shown) or by machining a helical cut into a cylinder. Opposing open ends 302 of the expandable helical spring 300 facilitate connection to spinous processes 10. FIG. 8b shows the same device as FIG. 8a, where the device has been stretched.

Another alternative for use as an elastic member in the device of the invention is depicted in FIG. 9. As shown, elastic members 400 and 400′ may be in the form of hollow, flattened tubes. The tube is elastically deformable with a degree of flexibility necessary to provide the desired clinical range of motion; e.g., substantially physiologic. A simple alternative adapter or connector in the form of, for example, a threaded male connector 404 and threaded female connector 410 can be used.

Another alternative for use as a connecting member in the device of the invention is depicted in FIG. 10. As shown, the elastic members are a substantially elastic pair of cords 500 and 500′. The cords can be connected by means of a threaded male end 504 and a correspondingly threaded female end 510, which may be crimped, swaged or otherwise attached to each cord to enable simple implantation and manufacture. Preferably, threaded female end 510 and threaded male end 504 are able to rotate without rotating cords 500 and 500′ to prevent cord twisting during insertion of male threaded end 504 into female threaded end 510.

Another alternative for use as a connecting member in the device of the invention is depicted in FIG. 11. As shown, the elastic members 600 and 600′ are in the form of elastic tethers, connectable by male adapter 152 and a retaining ring 160′ which has tabs which engage into groove 153.

Another alternative for use as an elastic connecting member in the device of the invention is depicted in FIG. 12. As shown, the connecting members 110 and 110′ are similar to connecting members 100 and 100′ described above except they connect more than two (in the illustrated case three) spinous processes 10.

Another alternative male connector 652 and female connector 660 are shown in FIG. 13. Male connector 652 incorporates a head 650 and a shaft 654 with a slot which forms two tines 653 and 653′. Female connector 660 incorporates a bore 662 and a recess 663. In operation, male connector 652 is guided into hole 12 in spinous process 10 until head 650 abuts spinous process 10. Female connector 660 is pushed onto the tip of male connector 652, thereby forcing together tines 653 and 653′ until they are able to pass through bore 662 at which point they return to their original positions and remain within recess 663.

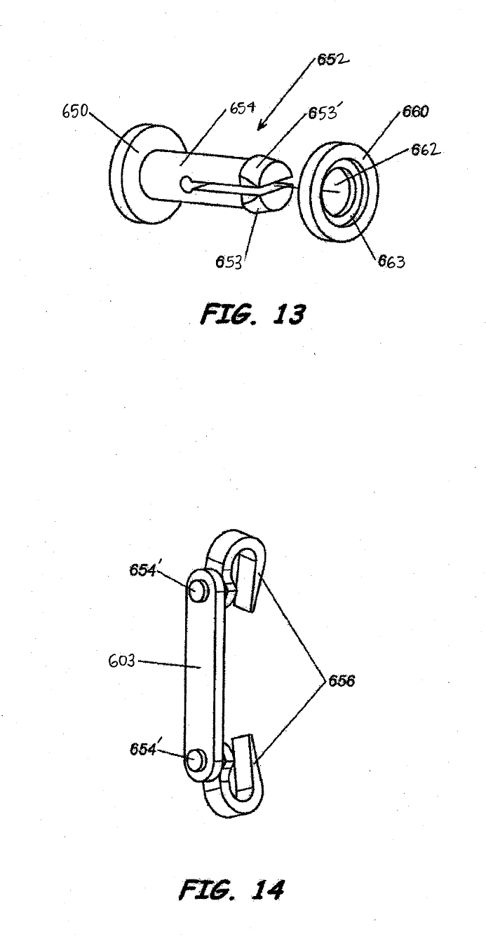

Another alternative embodiment of the invention is shown in FIG. 14. Elastic bridge member 603 is attached at each end to a hook 656 by attachment to a boss 654′. Each of hooks 656 may be anchored to bone; e.g., by placement of a bone screw through each hook into a lamina or spinous process of a vertebra.

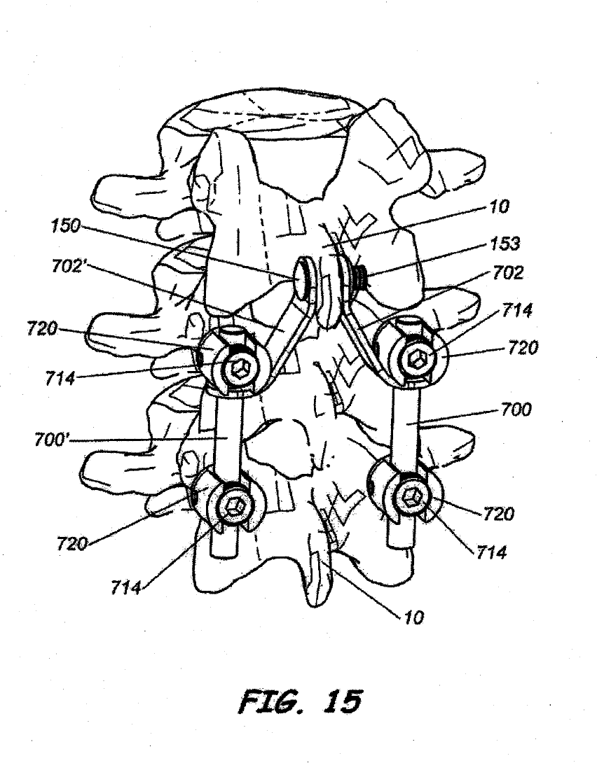

Another alternative embodiment of the invention is shown in FIG. 15. Here two vertebrae have been stabilized with rods 700 and 700′ by attachment to conventional pedicle screws 720 with connectors 714. Elastic elements 702 and 702′ are connected to rods 700 and 700′ and then connected to a spinous process 10 by means of a male connector 152 and female connector 160 in a fashion similar to the connectors discussed with respect to the embodiment of the invention shown in FIG. 3.

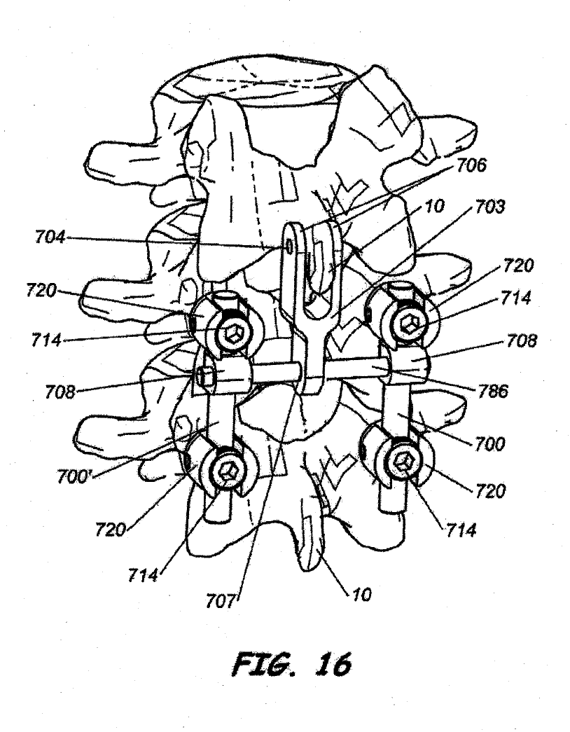

Another alternative embodiment of the invention is shown in FIG. 16. Here, two vertebrae have been stabilized with rods 700 and 700′ by attachment to conventional pedicle screws 720 with connectors 714. Crosslink 786 is attached to rods 700 and 700′ with crosslink connectors 708, installed as familiar to those of ordinary skill in the art. Elastic member 703 extends from and is rotatably or non-rotatably connected to crosslink 786; for example, by sliding crosslink 786 through hole 707 in elastic member 703. Many other attachments can be easily envisioned to attach elastic element to crosslink 786 including, without limitation, applying washers to secure the attachment at the center of the crosslink. Opposite its connection to crosslink 786, elastic element 703 splits into tines 706, which are placed on either side of spinous process 10. The tines are secured to spinous process 10 via any suitable connection; e.g., a male/female connection such as male connector 152 and female connector 160 as described with respect to the embodiment of the invention shown in FIG. 3.

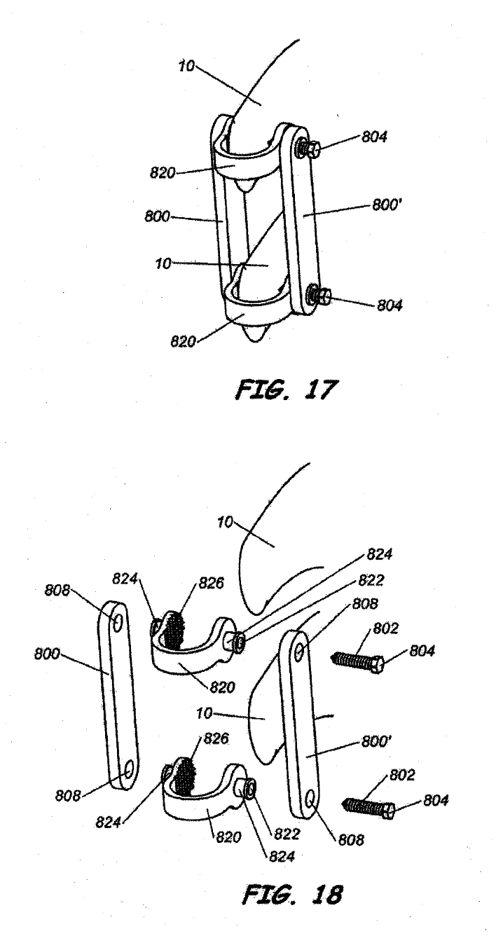

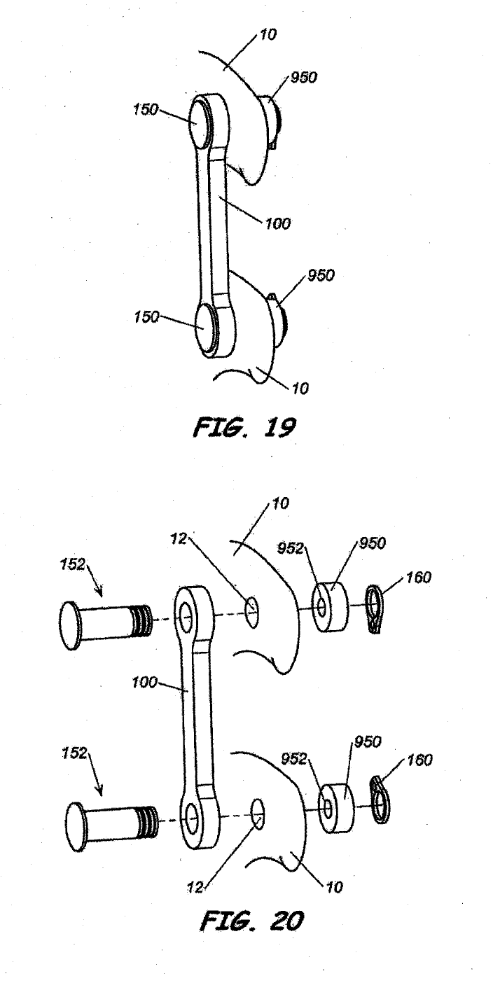

Another alternative embodiment of the invention is shown in FIGS. 17 and 18.

Clamps 820 are comprised of bosses 824, knurled surfaces 826 and threaded holes 822 to accommodate attachment screw 802. Each clamp 820 is connected to a spinous process 10 by turning hex head 804 of attachment screw 802, thereby threading it into threaded hole 822 and constricting clamp 820 onto spinous process 10. Elastic members 800 and 800′ are connected to bosses 824 of clamps 820.

Another alternative embodiment of the invention employing a single elastic member is shown in FIGS. 19 and 20. Elastic member 100 is attached to spinous processes 10 by placement of male connector 152 through a hole in elastic member 100, a hole 12 in spinous process 10, a hole 952 in a washer 950 and a bore in female connector 160. Female connector 160 is attached to male connector 152 as described above. Washer 950 is optional, if needed to further stabilize the attachment of elastic member 100 to spinous process 10.

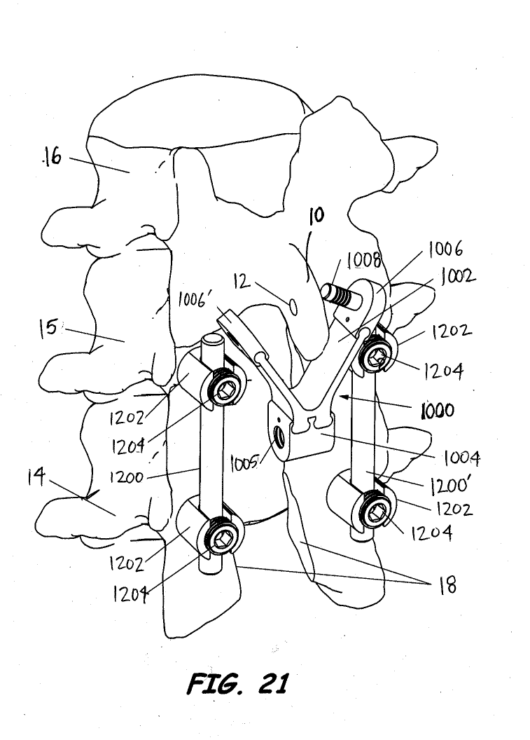

FIG. 21 depicts a perspective view of a posterior ligamentous stabilization and reconstruction device 1000 according to an embodiment of the invention, where the device is shown prior to or during surgical connection to a section of a subject's spine. As shown, device 1000 may include, for example, a flexible elastic member 1002 having a predetermined stiffness and elasticity and including first and second divergent leg portions 1002a, 1002b. Elastic member 1000 may be constructed from an elastic polymer material such as, for example but not limited to, silicone, urethane or other similar substantially elastic, durable, and biotolerated material. A first end of each leg portion may be positively coupled to a center lower end member 1004. A second end of each leg portion 1002a, 1002b, opposite the first end, may be positively coupled to a respective upper end member 1006, 1006′. End member 1006 includes a male connector 1008 such as, for example but not limited to, an elongated pin, formed integrally thereon and arranged to extend through a bore 12 in a spinous process 10 of a vertebra to attach the device 1000 thereto. Opposing end member 1006′ may be configured to receive and secure the pin 1008 on the opposite side of the spinous process 10 to connect the device 1000 to the spinous process 10 as discussed in further detail below. In FIG. 21, the device 1000 is shown adjacent first, second, and third vertebra 14, 15, 16, respectively. As shown, first and second vertebra 14, 15 have undergone a laminectomy 18, whereas third vertebra 16 has not. While third vertebra 16 is depicted as a supra-adjacent spinal segment in the embodiment depicted in FIG. 21, one of ordinary skill will recognize that vertebra 16 may be an adjacent or non-adjacent vertebra positioned above or below the treatment area. First and second vertebra 14, 15 have been stabilized (i.e., rigidly coupled or fused to one another) with pedicle screws 1202 and rods 1200, 1200′. Rods 1200, 1200′ are secured to pedicle screws 1202 with threaded locking caps 1204. A bore or hole 12 has been punched, bored, drilled or otherwise created in the spinous process 10 of third vertebra 16. The center (main) end member 1004 may include a bore 1005, which may be threaded.

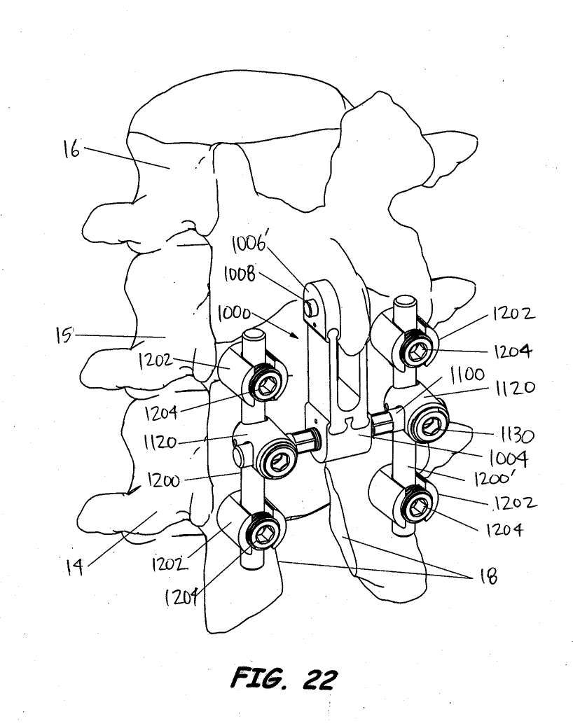

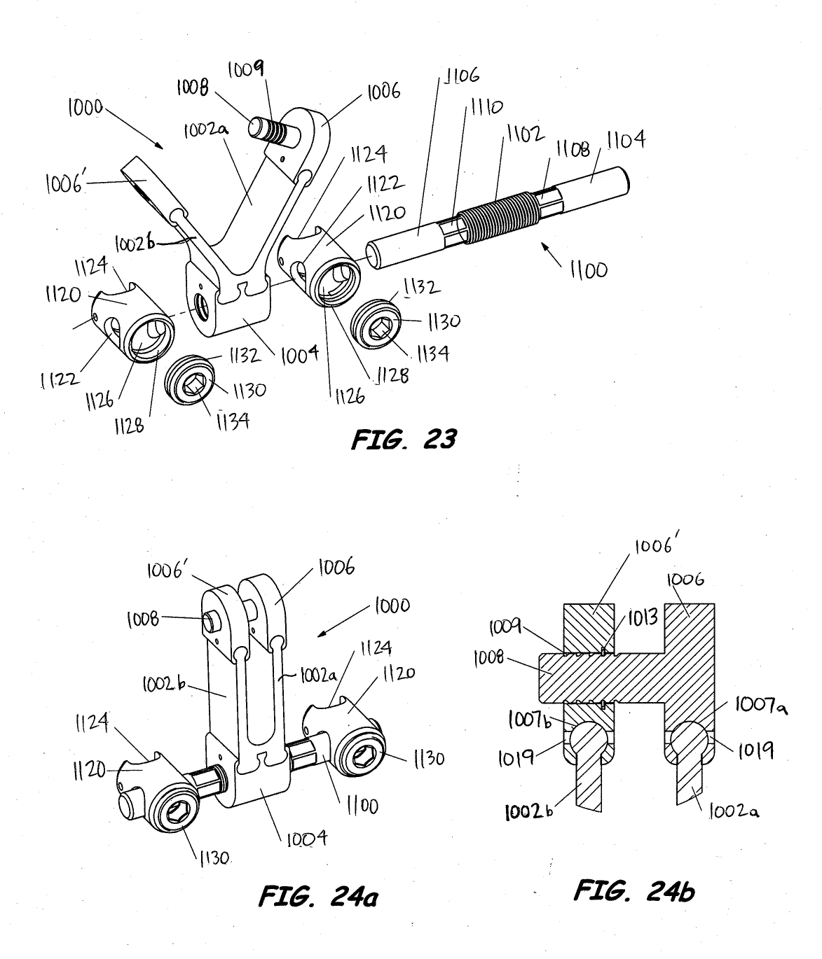

FIG. 22 illustrates a system enabling the attachment of device 1000 to the spinous process 10 of vertebra 16 and to rods 1200, 1200′ fixed to adjacent vertebra 14, 15. First, device 1000 is attached to vertebra 16 by sliding or pressing elongated pin 1008 of first upper end member 1006 through hole 12 in spinous process 10 and through receiving hole 1011 (see FIG. 26) in second upper end member 1006′. Next, a crosslink 1100 is threadedly extended through threaded bore 1005 in lower end member 1004 of device 1000. Respective ends 1104, 1106 (FIG. 23) of the crosslink 1100 are received in and secured by respective multiaxis connectors 1120 which removeably connect the crosslink 1100 to rods 1200, 1200′. When secured to spinous process 10 of vertebra 16 and to rods 1200, 1200′, the device 1000 may provide posterior ligamentous stabilization via elastic element 1002 which stabilizes the area of the spine being treated while preserving a desirable range of motion (e.g., a substantially physiologic range of motion).

An embodiment of the device 1000 is illustrated in further detail in FIGS. 23, 24a, 24b, 25 and 26. FIG. 23, for example, depicts an exploded perspective view of a system including the device 1000 shown in FIGS. 21-22 as well as crosslink 1100 and multiaxis connectors 1120. FIG. 24a depicts a perspective view of the system shown in FIG. 23 in an assembled and connected state. As shown in the illustrative embodiment depicted in FIGS. 23, 24a, and 24b, the device 1000 includes an elastic member 1002 having a predetermined stiffness and elasticity and including first and second divergent leg portions 1002a, 1002b. Respective ends 1007a, 1007b of leg portions 1002a, 1002b are fixedly connected to the end members 1006, 1006′, for example, by being molded or otherwise rigidly fixed within complimentary recesses 1017a, 1017b defined in a lower part of the end members 1006, 1006′ (see FIG. 24b). End members 1006, 1006′ may have one or more small holes 1019 formed proximate recesses 1017a, b such that, during molding, the material forming elastic member 1002 may flow in and through holes 1019 to positively fix ends 1007a, 1007b within recesses 1017a, b. Alternatively, or additionally, protrusions or other elements (not shown) may be provided in recesses 1017a, b such that, during molding, the material forming elastic member 1002 may flow around such elements to positively fix ends 1007a, 1007b within recesses 1017a, b.

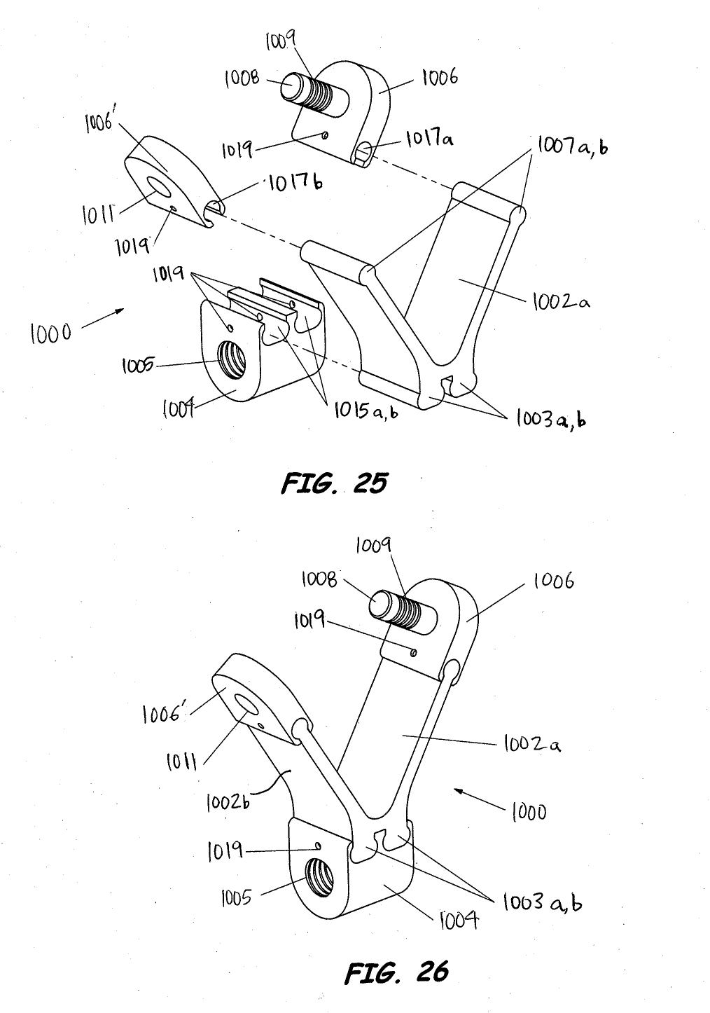

Likewise, lower ends (ribs) 1003a,b of leg portions 1002a, b may be fixedly connected to the lower end member 1004, for example, by being molded or otherwise rigidly fixed within complimentary recesses 1015a, 1015b defined in an upper part of the lower end member 1004. One or more small holes 1019 may also be formed proximate recesses 1015a, b such that, during molding, the material forming elastic member 1002 may flow in and through holes 1019 to positively fix ends 1003a,b within recesses 1015a, b. Alternatively, or additionally, protrusions or other elements (not shown) may be provided in recesses 1015a, b such that, during molding, the material forming elastic member 1002 may flow around such elements to positively fix ends 1003a, 1003b within recesses 1015a, b. The exploded view of FIG. 25 shows assembly of device 1000 by sliding the elastic member ends 1003a, b and 1007a, b into the corresponding recesses 1015a, b and 1017a, b, respectively. In such case, locking holes 1019 could, for example, accommodate pins (not shown) to lock elastic member 1002 in respective recesses. Alternatively, elastic member 1002 is molded to the end members 1004, 1006, 1006′ and the locking holes 1019 are filled with elastic material during molding to prevent elastic member 1002 from disengaging from the end members.

End member 1006 includes an elongated pin 1008 formed integrally thereon and having, for example, a grooved outer surface 1009. The pin 1008 is arranged and configured to extend through a bore 12 in a spinous process 10 of a vertebra to attach the device 1000 thereto. Opposing end member 1006′ may be configured to receive and secure the male connector (pin) 1008 on the opposite lateral side of the spinous process 10 to connect the device 1000 to the spinous process 10. For example, upper end member 1006′ may have a female connector 1011 such as, for example, a through hole which contains a retaining ring 1013 (see FIG. 24b) configured to be captured and retained in a ratchet groove 1009 on pin 1008. Ratchet grooves 1009 on elongated pin or boss 1008 on first upper end member 1006 engage retaining ring 1013 so that retaining ring 1013 allows elongated pin 1008 to move through receiving hole 1011 in one direction only. Therefore, retaining ring 1013 captures pin 1008 and maintains attachment of device 1000 to the spinous process 10 of vertebra 16.

As shown in FIG. 23, crosslink 1100 may be in the form of a substantially cylindrical rod having end portions 1104, 1106 and a central, externally threaded portion 1102 configured to be received in and attached to lower end member 1004 of the device 1000 by engaging external threads 1102 with internally threaded bore 1005 on the lower end member 1004. Crosslink 1100 may also include hex features 1108, 1110 arranged to be manipulated by a tool (not shown) to facilitate threaded engagement.

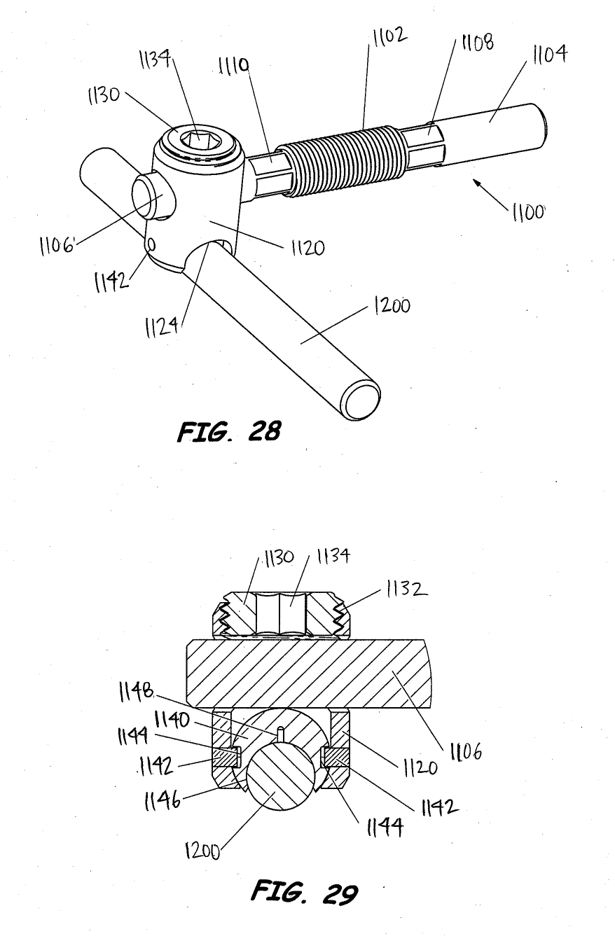

Crosslink 1100 is attached to rods 1200, 1200′ with multiaxis connectors 1120, shown in further detail in FIGS. 28 and 29. Multiaxis connector housing 1120 includes a central bore 1126 with internal threads 1128, and a transverse bore 1122 which may extend completely through multiaxis connector housing 1120 perpendicular to the axis of bore 1126. A recess 1124 may be provided opposite threaded end 1128, the recess configured to receive one of the rods 1200, 1200′ at a range of angles including perpendicular to both the central bore 1126 and the transverse bore 1122. A set screw 1130 may be provided having threads 1132 configured to engage internal threads 1128 of the multiaxis connector housing 1120. Tightening of the set screw 1130 via tool receiving portion (e.g., hex head) 1134 serves to secure an end 1104, 1106 of the crosslink 1100 within the multiaxis connector housing 1120.

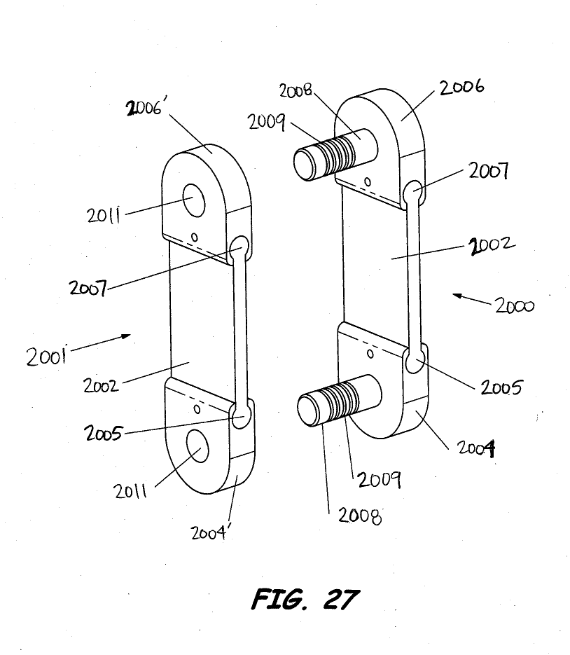

FIG. 27 depicts a perspective view of a posterior ligamentous stabilization and reconstruction device according to another embodiment of the invention, the device including two separate elements 2000, 2001. As shown in the alternative embodiment of FIG. 27, a crosslink is not used to secure the invention to the spine. In this regard, the embodiment may more closely resemble the embodiments shown and described in FIGS. 1-14. As shown in FIG. 27, the device includes first and second parallel elements 2000, 2001. The first element 2000 includes an elastic member 2002 and two end members 2004, 2006 connected at respective ends thereof. Ends 2005, 2007 of the elastic member 2002 may be coupled to the end members 2004, 2006 in a sufficient manner as described above with reference to the embodiment depicted in FIGS. 25-26. The second element 2001 also includes an elastic member 2002 and two end members 2004′, 2006′ connected at respective ends thereof. Ends 2005, 2007 of the elastic member 2002 may be coupled to the end members 2004, 2006 as previously described. Each of end members 2004 and 2006 may include an integrally formed pin 2008 with grooves 2009 similar to the pin 1008 described above. Each of end members 2004′,2006′ may include a receiving through hole 2011 constructed similar to hole 1011 described above. Connection of the elements 2000,2001 together may be substantially similar to the connection shown in FIG. 24b, and connection to the spine may utilize holes 12 in the spinous processes 10 of two adjacent or non-adjacent vertebrae.

FIG. 28 depicts a perspective view of the crosslink 1100 and multiaxis connector 1120 of FIGS. 22, 23, and 24a and as coupled to rod 1200. FIG. 29 depicts a cross-sectional view of the multiaxis connector 1120 secured to the crosslink 1100 and rod 1200, the rod 1200 and crosslink 1100 extending substantially normal to one another. As illustrated in FIG. 29, a spherical ball 1140 may be positioned within the multiaxis connector housing 1120 by engagement of pins 1142 into corresponding slots 1144 in ball 1140. This may prevent spherical ball 1140 from falling out of multiaxis connector housing 1120, and may also serve to limit rotation of spherical ball 1140. Spherical ball 1140 may also include a cylindrical recess 1146 sized to engage with rods 1200, 1200′, and a flexure cut 1148 designed to make spherical ball 1140 flexible so that cylindrical recess 1146 can flex open and receive rod 1200, 1200′. Cylindrical recess 1146 may be cut and configured so that when it engages rod 1200, 1200′, it contacts more than half of the perimeter of rod 1200, 1200′, as shown in the cross-sectional view depicted in FIG. 29. As set screw 1130 is rotatably tightened within multiaxis connector housing 1120, end portion 1106 of cross connector 1100 is forced onto spherical ball 1140, which in turn is forced down into a concave spherical seat of central bore 1126, locking spherical ball 1140 in place and also locking it onto rod 1200.

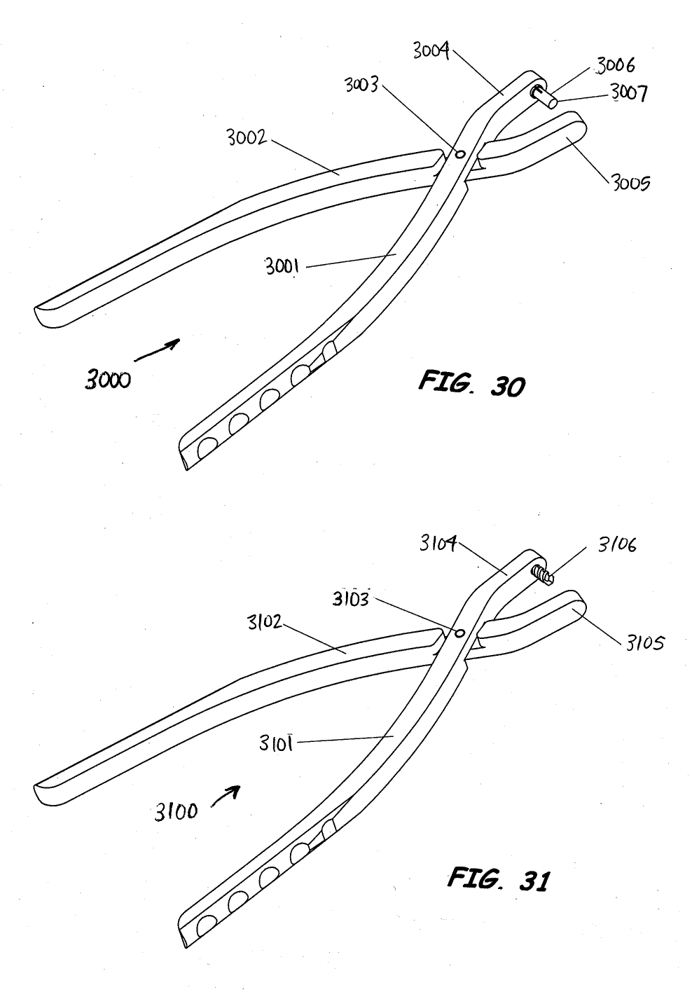

FIGS. 30 and 31 depicts several possible embodiments of a tool 3000, 3100 constructed and configured to form a bore through a spinous process(es) of a subject. Creation of hole 12 in a spinous process(es) 10 (see FIG. 21) may be accomplished with an instrument or tool 3000, 3001 such as those depicted in FIGS. 30 & 31. In FIG. 31, a punch 3006 may be provided including a sharp, hollow cutting edge 3007 attached to pliers which are formed from a first handle 3001 and a second handle 3002 pivotably coupled to one another about a pivot point (pin) 3003. Punch 3006 may be manually driven through the spinous process 10 by gripping handles 3001 and 3002 until punch 3006 is forced entirely through the spinous process 10 and contacts a guard 3005. In an alternative embodiment illustrated in FIG. 31, instead of a punch, a drill bit 3106 may be attached to the pliers. The spinous process 10 is gripped as before, but instead of driving drill bit straight through, handles 3101 and 3102 are rotated about the axis of drill bit 3106 in a back-and-forth arc until drill bit 3106 bores completely through the spinous process and contacts guard 3105. Drill bit 3106 could also be electrically or pneumatically powered to rotate when activated.

Methods for implanting and deploying the devices of the invention include the following steps for use in (i) stabilizing adjacent bones; (ii) connecting adjacent vertebral levels; and/or (iii) preventing or treating kyphosis, listhesis, or segmental spinal instability in a subject in need of treatment thereof. In each such method, the steps comprise: forming two holes through adjacent spinous processes, attaching clamps to adjacent spinous processes, or forming one hole through an adjacent spinous process (or clamp) if connecting directly to spinal instrumentation (e.g. crosslink, rod, pedicle screw, laminar screw, lateral mass screw, etc.); connecting each end of a connecting member into the appropriate element of a connectable adapter (male or female element), wherein the connecting member is preferably pre-selected to provide a clinically appropriate level of stiffness; and joining the adapter elements through the spinous processes, or between one spinous process and an adjacent spinal instrumentation construct (e.g. crosslink, rod, pedicle screw, laminar screw, lateral mass screw, etc.), to secure the connecting members onto the spinous processes or onto adjacent spinal instrumentation.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The subject treated by the presently disclosed methods and devices in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The terms “treat” or “treating,” and grammatical derivatives thereof, as used herein refer to any type of treatment that imparts a benefit to a subject afflicted with a disease or illness, including any measurable improvement in the condition of the subject (e.g., in one or more symptoms), reducing a symptom of the condition, inhibiting an underlying cause or mechanism related to the condition, delay in the progression of the condition, prevention or delay of the onset of the disease or illness, e.g., prophylactic treatment, enhancement of normal physiological functionality, and the like.

The term “effective,” as that term is used in the specification and/or claims, means adequate to accomplish a desired, expected, or intended result, e.g., to prevent, alleviate, or ameliorate symptoms of disease or prolong the survival of the subject being treated.

Following long-standing patent law convention, the terms “a,” “an,” and “the” refer to “one or more when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be nonlimiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references are herein incorporated by reference to the same extent as if each publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A spinal stabilization device comprising:
an elastic member having an elongate aspect for spanning a distance between a pair of adjacent spinous processes, the elastic member comprising a first end and a second end and having a predetermined tensile stiffness; and
a plurality of connection elements adapted to attach respective ends of the elastic member to the spinous processes, wherein the connection elements are configured to be passed through a bore in the spinous process, and wherein the stiffness of the elastic member is selected to provide a clinically appropriate and substantially physiologic range of motion.

2. The device of claim 1, wherein the elastic member is selected from the group consisting of a helical spring, a flexure, an elastic cord, an elastic plate, and an elastic tube.

3. The device of claim 1, wherein the connection elements comprises a male connector and a corresponding female connector configured to receive and retain the male connector.

4. The device of claim 1, wherein the elastic member comprises a plurality of elastic members, and wherein the elastic members are positioned on opposing sides of the spinous process and coupled to one another through the bore in the spinous process by the connection elements.

5. The device of claim 4, wherein ends of each elastic member are substantially permanently secured to end members on which the connection elements are integrally formed.

6. The device of claim 5, wherein one of the plurality of elastic members is secured to first end members having an integrally formed male connector.

7. The device of claim 6, wherein the other of the plurality of elastic members is secured to second end members having an integrally formed female connector configured to receive and retain the male connector.

8. A spinal stabilization system comprising:
an elastic member having a predetermined tensile stiffness;
a connection element coupled to an end of the elastic member and adapted to attach the elastic member to a spinous process of a subject's spine, wherein the connection element is adapted to be passed through a bore in the spinous process;
a rigid rod configured for attachment to the spine; and
a further connection element for connection of the elastic member to the rigid rod, wherein the predetermined tensile stiffness of each elastic member is selected to provide a clinically appropriate and substantially physiologic range of motion.

9. The system of claim 8, wherein the further connection element comprises a crosslink member configured to be removeably coupled to a plurality of the rigid rods, and wherein the crosslink member removeably engages a lower end member secured to an end of the elastic member.

10. The system of claim 8, wherein the rigid rod is configured to be fixedly attached to the spine with at least one pedicle screw, lateral mass screw, or laminar hook for attachment to the spine.

11. The system of claim 8, wherein the elastic member comprises first and second divergent leg portions.

12. The system of claim 11, wherein the connection element comprises a plurality of connection elements coupled to respective ends of the first and second divergent leg portions.

13. The system of claim 12, wherein the divergent leg portion ends are substantially permanently secured to end members on which the connection elements are integrally formed.

14. The system of claim 13, wherein one of the divergent leg portions of the elastic member is secured to a first end member having an integrally formed male connector.

15. The system of claim 14, wherein the other of the divergent leg portions is secured to a second end member having an integrally formed female connector configured to receive and retain the male connector.

16. The system of claim 15, wherein another end of each of the divergent leg portions is substantially permanently secured to a lower end member.

17. The system of claim 16, wherein the further connection element comprises a crosslink member configured to be removeably coupled to a plurality of the rigid rods, and wherein the crosslink member removeably engages the lower end member secured to the elastic member.

18. The system of claim 17, wherein the further connection element comprises a multiaxis connector configured to couple the crosslink member to the rigid rods.

19. The system of claim 18, wherein the multiaxis connector comprises:
a housing including
a first bore extending along a first axis and configured to receive an end of the crosslink member;
a second bore extending along a second axis substantially perpendicular to the first axis, wherein the second bore is configured to receive a fastener member to secure the crosslink member in the first bore; and
a recess defined at an end of the housing and extending substantially perpendicular to the first and second axes, wherein the recess is configured to receive the rigid rods.

20. The system of claim 19, wherein the multiaxis connector further comprises:
a substantially spherical ball retained within the housing adjacent the recess, wherein the ball includes a substantially cylindrical recess configured to receive one of the rigid rods.

21. The system of claim 20, wherein the multiaxis connector further comprises at least one pin engaging a corresponding slot on the ball to retain the ball and limit rotation thereof.

22. The system of claim 20, wherein the spherical ball further comprises a flexure cut configured to make the spherical ball flexible so that cylindrical recess can flex open and receive the rigid rod.
